SEQUENCE LISTING (6 sequences)

SEQ ID NO: 1 - DNA, 229 bp, Artificial Sequence
  Sleeping Beauty ITR 5′
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagt

SEQ ID NO: 2 - DNA, 229 bp, Artificial Sequence
  sleeping beauty ITR 3′
attgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 3 - DNA, 309 bp, Artificial Sequence
  piggyBac ITR 5′
ccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 4 - DNA, 238 bp, Artificial Sequence
  piggybac ITR 3′
taaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg

SEQ ID NO: 5 - DNA, 35 bp, Artificial Sequence
  piggybac minimal ITR
ccctagaaagatagtctgcgtaaaattgacgcatg

SEQ ID NO: 6 - DNA, 63 bp, Artificial Sequence
  piggybac minimal ITR 3′
catgcgtcaattttacgcatgattatctttaacgtacgtcacaatatgattatctttctaggg

(19) United States
(12) Patent Application Publication
(10) Pub. No.: US 2016/0046959 A1
(43) Pub. Date: Feb. 18, 2016

(54) REPRODUCIBLE METHOD FOR TESTIS-MEDIATED GENETIC MODIFICATION (TGM) AND SPERM-MEDIATED GENETIC MODIFICATION (SGM)

(71) Applicants: Carlisle P. LANDEL, Lexington, KY (US); Eric . OSTERTAG, (US); Joseph RUIZ, Cary, NC (US); Tseten YESHI, Lexington, KY (US); TRANSPOSAGEN BIOPHARMACEUTICALS, INC., Lexington, KY (US)

(72) Inventors: Carlisle P. Landel, Lexington, KY (US); Eric M. Ostertag, Lexington, KY (US); Joseph Ruiz, Cary, NC (US); Tseten Yeshi, Lexington, KY (US)

(21) Appl. No.: 14/776,656

(22) PCT Filed: Mar. 17, 2014
(86) PCT No.: PCT/US14/30546

§ 371 Date: Sep. 14, 2015

Related U.S. Application Data
(60) Provisional application No. 61/802,195, filed on Mar. 15, 2013.

(51) Int. Cl. C12N 15/85; A01K 67/027

(52) U.S. Cl. CPC C12N 15/8509; A01K 67/0275; A01K 2217/05; C12N 2800/40; C12N 2800/90; C12N 2999/007

(57) ABSTRACT

The present invention provides a method of direct germline mutagenesis of a non-human animal.

### FIELD OF THE INVENTION

The present invention is directed, in part, to methods of integrating nucleic acids encoding genes of interest into the chromosoma 1 DNA of an animal, nucleic acids prepared for the same, compositions comprising such nucleic acids, and kits comprising such nucleic acids, and non-human transgenic animals mutagenized by these methods.

### BACKGROUND OF THE INVENTION

The production of transgenic animals is a complex process, requiring the production, culture and manipulation of large numbers of embryos into which DNA is inserted via microinjection, via lentiviral infection, via combination with genetically modified embryonic or induced pluripotent stem cells, or via the intracytoplasmic injection of DNA bound to disrupted sperm. The process is technically demanding and requires the use of large numbers of animals for embryo production, and utilizes complex and expensive equipment. An attractive theoretical alternative would be to somehow utilize the sperm to carry exogenous DNA into the embryo. This has led to the a large number of reports being published on the techniques of testis-mediated gene transfer (TMGT), wherein DNA is somehow applied to the testis of animals who then transfer this DNA to their offspring, or sperm-mediated germ transfer (SMGT), where DNA is applied to sperm which is then used to fertilized oocytes in vitro or in vivo (for review, see Parrington et al., 2011; Niu and Lang, 2008; and Coward et al., 2007).

There are multiple reports in the literature concerning the ability to produce transgenic animals via the injection of DNA solutions of various kinds directly into the testis. An initial report by Sato et al (1995) demonstrated that injecting a calcium phosphate precipitate of plasmid DNA directly into the testis of mice resulted in the detection of the injected DNA in isolates of sperm from the epididymis or from the uteri of females mated with the injected males, but that the DNA could not be detected in embryos. Since that initial report, many reports have been published describing the injection of DNA into the testis (in combination with liposomes, with electroporation, or with viral vectors) and the subsequent mating of these animals to produce F1 animals in which the transgene can be detected, but only a two reports demonstrated that the transgene is transmitted to the F2 generation when these initial founder animals are bred further (Sato et al. 1999; Miao and Zhang, 2011). At best, successful, stable, germline integration of the transgene must be regarded as an extremely low-efficiency event (Parrington et al., 2011).

Many of the studies attempting this method of testis-mediated gene transfer (TMGT) utilized a reporter gene such as GFP in the transgene construct in order to track the expression of the transgene, and indeed, the reporter gene is seen to be expressed in embryos resulting from the cross. For example, Yonezawa et al (2001) show fluorescent morula and report fluorescent 14-day fetuses when a GFP transgene was injected into the testis in conjunction with liposomes. Unremarked in all these papers was the profound implication of this demonstrated embryonic expression of transgenes delivered by TMGT: if the transgene delivered by this method can function, then were this transgene to encode a transposase, and if it were delivered in conjunction with a target transposon, then a transposon/transposase system could be harnessed to drive stable, germline transgene integration. We therefore set out to accomplish this.

The piggyBac transposon system was originally identified as a Lepidopteran transposon and since has been adapted as an efficient vector for inserting DNA into the genome of cells and embryos (Ding et al 2005). PiggyBac inserts into TTAA sites in the genome and is unique in its ability to integrate very large (>100 kb) fragments of DNA into the genome and in its “footprint-free” excision from the genome.

### SUMMARY OF THE INVENTION

Using a mixture of plasmids, one carrying a piggyBac transposon and one carrying a hyperactive piggyBac transposase gene driven by a CMV promoter, mixed with lipofection reagents and injected into either the seminiferous tubules via the rete testis or into the body of the testis, we were able to obtain germline transgenic animals with high efficiency.

The present invention also provides cells comprising any of the nucleic acids or vectors described herein. In some embodiments, the cell is a sperm cell within the animal disclosed herein.

The present invention also provides kits comprising: a vector comprising a nucleic acid encoding any of the proteins described herein; and a transposon comprising an insertion site for an exogenous nucleic acid, wherein the insertion site is flanked by a first inverted repeat sequence comprising a sequence at least about 90% sequence identity to an inverted terminal repeat (ITR) of any transposon known in the art and/or a second inverted repeat sequence comprising a sequence at least about 90% sequence identity to the reverse sequence of any ITR of any transposon known in the art.

The present invention also provides non-human, transgenic animals comprising a nucleic acid molecule encoding any of the proteins described herein. In some embodiments, the non-human, transgenic animal further comprises a transposon comprising an insertion site for an exogenous nucleic acid, wherein the insertion site is flanked by a first inverted repeat sequence and/or a second inverted repeat sequence.

The present invention also provides methods of integrating an exogenous nucleic acid into the genome of at least one cell of a multicellular or unicellular organism comprising administering directly to the multicellular or unicellular organism: a composition comprising a transposon comprising an exogenous nucleic acid, wherein the exogenous nucleic acid is flanked by a sequence at least about 90% sequence identity to a first inverted repeat sequence and/or a sequence at least about 90% sequence identity to a second inverted repeat sequence of a transposon; and a hyperactive transposase protein described herein to excise the exogenous nucleic acid from a plasmid, episome, or transgene and integrate the exogenous nucleic acid into the genome. In some embodiments, the protein is administered as a nucleic acid encoding the protein. In some embodiments, the transposon and nucleic acid encoding the protein are present on separate vectors. In some embodiments, the transposon and nucleic acid encoding the protein are present on the same vector. In some embodiments, the multicellular or unicellular organism is a vertebrate. In some embodiments, the vertebrate animal is a non-human mammal. In some embodiments, the vertebrate animal is a rodent, bovine, equine or other domesticated animal species. In some embodiments, the exogenous nucleic acid comprises a gene.

The present invention also provides methods of generating a non-human, transgenic animal comprising a germline mutation comprising: injection of a composition into the testis of animal, the composition comprising: (i) a nucleic acid sequence comprising an exogenous gene flanked by an ITR of a transposase; and (ii) a nucleic acid sequence encoding a transposon. In some embodiments, the exogenous nucleic acid sequence is not flanked by the ITR of a transposase, but, in such embodiments, the composition comprises a nucleic acid sequence that encodes one or more of the enzymes disclosed herein except a transposase.

The present invention also provides methods of generating a non-human, transgenic animal comprising: introducing a nucleic acid molecule encoding any of the proteins described herein into a cell of the testis via direct needle injection. In some embodiments, the composition is pyrogen-free.

### DESCRIPTION OF EMBODIMENTS

As used herein, “sequence identity” is determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety).

As used herein, “conservative” amino acid substitutions may be defined as set out in Tables A, B, or C below. Hyperactive transposases and transposases include those wherein conservative substitutions have been introduced by modification of polynucleotides encoding polypeptides of the invention. Any sequences disclosed herein can be modified by conservative amino acid substitutions and are contemplated by the invention. In some embodiments the transposase or hyperactive transposases comprise one or more conservative substitutions but retain their function as transposases. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A.

TABLE A
Conservative Substitutions I

| Side Chain Characteristics | Amino Acid |
|---|---|
| Aliphatic | |
| Non-polar | G A P I L V F |
| Polar-uncharged | C S T M N Q |
| Polar-charged | D E K R |
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | Amino Acid |
|---|---|
| Non-polar (hydrophobic) | |
| Aliphatic: | A L I V P. |
| Aromatic: | F W Y |
| Sulfur-containing: | M |
| Borderline: | G Y |
| Uncharged-polar | |
| Hydroxyl: | S T Y |
| Amides: | N Q |
| Sulfhydryl: | C |
| Borderline: | G Y |
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

As used herein, “more than one” of the aforementioned amino acid substitutions means 2, 3, 4, 5, 6, 7, 8, 9, or 10 of the recited amino acid substitutions. In some embodiments, “more than one” means 2, 3, 4, or 5 of the recited amino acid substitutions. In some embodiments, “more than one” means 2, 3, or 4 of the recited amino acid substitutions. In some embodiments, “more than one” means 2 or 3 of the recited amino acid substitutions. In some embodiments, “more than one” means 2 of the recited amino acid substitutions.

The present invention also provides nucleic acids encoding any one of the hyperactive transposase proteins described herein. Thus, the present invention provides nucleic acids encoding a protein that comprises at least 75% (or 80%, 85%, 90%, 95%, or 99%) sequence identity to known ITRs and transposase sequences. The present invention also provides nucleic acids encoding a protein that comprises at least 75% (or 80%, 85%, 90%, 95%, or 99%) sequence identity to known ITRs and transposase sequences.

As used herein, the terms “fragment” and “fragment of a transposon” are meant to refer to DNA sequences which are not complete transposon DNA sequences (i.e. full-length DNA sequences) but DNA sequences shorter in length than the full-length sequence which consist of nucleotide sequences identical to nucleotide sequences of portions of a full-length DNA sequence of a transposon. A fragment of a transposon may function like a full length DNA. In some embodiments, a fragment of a transposon is a truncated form of the wild-type or full-length DNA transposon sequence. In some embodiments a fragment of a transposon is an internal tandem repeat of the transposon. For example, in some embodiments where compositions or methods comprise transplanted haplotypes, the haplotypes comprise fragments of full-length transposons that flank transgenes of mutated genes of interest. In some embodiments, the transplanted haplotypes comprise at least one or more of any combination of the fragments of a transposon comprising the following DNA sequences:

TABLE 1
Sleeping Beauty 5′ ITR:
CAGTTGAAGTCGGAAGTTTACATACACTTAAGTTGGAGTCATTAAAAC
TCGTTTTTCAACTACTCCACAAATTTCTTGTTAACAAACAATAGTTTT
GGCAAGTCAGTTAGGACATCTACTTTGTGCATGACACAAGTCATTTTT
CCAACAATTGTTTACAGACAGATTATTTCACTTATAATTCACTGTATC
ACAATTCCAGTGGGTCAGAAGTTTACATACACTAAGT
Sleeping Beauty 3′ ITR:
ATTGAGTGTATGTAAACTTCTGACCCACTGGGAATGTGATGAAAGAAA
TAAAAGCTGAAATGAATCATTCTCTCTACTATTATTCTGATATTTCAC
ATTCTTAAAATAAAGTGGTGATCCTAACTGACCTAAGACAGGGAATTT
TTACTAGGATTAAATGTCAGGAATTGTGAAAAAGTGAGTTTAAATGTA
TTTGGCTAAGGTGTATGTAAACTTCCGACTTCAACTG
PiggyBac 5′ ITR:
CCCTAGAAAGATAGTCTGCGTAAAATTGACGCATGCATTCTTGAAATA
TTGCTCTCTCTTTCTAAATAGCGCGAATCCGTCGCTGTGCATTTAGGA
CATCTCAGTCGCCGCTTGGAGCTCCCGTGAGGCGTGCTTGTCAATGCG
GTAAGTGTCACTGATTTTGAACTATAACGACCGCGTGAGTCAAAATGA
CGCATGATTATCTTTTACGTGACTTTTAAGATTTAACTCATACGATAA
TTATATTGTTATTTCATGTTCTACTTACGTGATAACTTATTATATATA
TATTTTCTTGTTATAGATATC
(minimal sequence is underlined and bold, i.e.,
first 35 bp)
PiggyBac 3′ ITR:
TAAAAGTTTTGTTACTTTATAGAAGAAATTTTGAGTTTTTGTTTTTTT
TTAATAAATAAATAAACATAAATAAATTGTTTGTTGAATTTATTATTA
GTATGTAAGTGTAAATATAATAAAACTTAATATCTATTCAAATTAATA
AATAAACCTCGATATACAGACCGATAAAACACATGCGTCAATTTTACG
CATGATTATCTTTAACGTACGTCACAATATGATTATCTTTCTAGGG
(minimal sequence is underlined and bold, i.e.,
first 35 bp)
Minimal 5′ piggyBac ITR
CCCTAGAAAGATAGTCTGCGTAAAATTGACGCATG
Minimal 3′ piggyBac ITR
CATGCGTCAATTTTACGCATGATTATCTTTAACGTACGTCACA
ATATGATTATCTTTCTAGGG

In some embodiments, the only transposon fragment in the transplanted haplotype consists of a PiggyBac 5′ ITR and a PiggyBac 3′ ITR. In some embodiments, the only transposon fragment in the transplanted haplotype consists of a Sleeping Beauty 5′ ITR and a Sleeping Beauty 3′ ITR. In some embodiments, the transplanted haplotype comprises a transgene flanked by a PiggyBac 5′ ITR and a PiggyBac 3′ ITR. In some embodiments, the transplanted haplotype comprises a transgene flanked by a Sleeping Beauty 5′ ITR and a Sleeping Beauty 3′ ITR. In some embodiments, the transplanted haplotype comprises a transgene flanked by the following sequences: 5′ CCCTAGAAAGATAGTCTGCGTAAAATTGACGCATG and on the 3′ end (from 5′ to 3′): CATGCGTCAATTTTACGCATGATTATCTTTAACGTACGTCACAATATGATTATC TTTCTAGGG. In some embodiments, the transplanted haplotype comprises a hyperactive transposon.

The present invention also provides vectors comprising any of the aforementioned nucleic acids. Thus, the present invention provides vectors comprising a nucleic acid that encodes a protein that comprises at least 75% (or 80%, 85%, 90%, 95%, or 99%) sequence identity to the transposase sequences and other enzymes provided herein.

In some embodiments, the vector is a plasmid. In other embodiments, the vector is not a retrovirus. In some embodiments, the vector is a linear DNA molecule.

The present invention also provides cells or organisms comprising any of the aforementioned nucleic acids.

In some embodiments, the cells or organisms comprise a nucleic acid that encodes a protein that comprises or possesses at least 75% (or 80%, 85%, 90%, 95%, or 99%) sequence identity to any nucleic acid sequence disclosed herein.

In some embodiments, the cell comprises any of the vectors disclosed herein.

The present invention also provides kits comprising: 1) any of the vectors disclosed herein in one or multiple containers comprising a restriction enzyme site for ligation of a heterologous gene into one or more expression constructs disclosed herein; and 2) a nucleic acid sequence comprising an enzyme, wherein the enzyme is chosen from: a transposase or hyperactive transposase.

The transposons described herein can include a wide variety of inserted nucleic acids, where the nucleic acids can include a sequence of bases that is endogenous and/or exogenous to a multicellular or unicellular organism. The nature of the nucleic acid can vary depending upon the particular protocol being carried out. In some embodiments, the exogenous nucleic acid can be a gene. The inserted nucleic acid that is positioned between the flanking inverted repeats can vary greatly in size. The only limitation on the size of the inserted nucleic acid is that the size should not be so great as to inactivate the ability of the transposon system to integrate the transposon into the target genome. The upper and lower limits of the size of inserted nucleic acid can be determined empirically by those of skill in the art. In some embodiments, the transposons of the invention include those transposase sequences identified and disclosed in Human Gene Therapy 23:311-320 (March 2012). In some embodiments, the methods include any of the steps disclosed in Belay et. al. Pages 406-413 (8) of Current Gene Therapy.

In some embodiments, the inserted nucleic acid comprises at least one transcriptionally active gene, which is a coding sequence that is capable of being expressed under intracellular conditions, e.g. a coding sequence in combination with any requisite expression regulatory elements that are required for expression in the intracellular environment of the target cell whose genome is modified by integration of the transposon. The transcriptionally active genes of the transposon can comprise a domain of nucleotides, i.e., an expression module that includes a coding sequence of nucleotides operably linked with requisite transcriptional mediation or regulatory element(s). Requisite transcriptional mediation elements that may be present in the expression module include, but are not limited to, promoters, enhancers, termination and polyadenylation signal elements, splicing signal elements, and the like.

In some embodiments, the expression module includes transcription regulatory elements that provide for expression of the gene in a broad host range. A variety of such combinations are known, where specific transcription regulatory elements include, but are not limited to: SV40 elements, transcription regulatory elements derived from the LTR of the Rous sarcoma virus, transcription regulatory elements derived from the LTR of human cytomegalovirus (CMV), hsp70 promoters, and the like.

In some embodiments, at least one transcriptionally active gene or expression module present in the inserted nucleic acid acts as a selectable marker. A variety of different genes have been employed as selectable markers, and the particular gene employed in the vectors described herein as a selectable marker is chosen primarily as a matter of convenience. Known selectable marker genes include, but are not limited to: thymidine kinase gene, dihydrofolate reductase gene, xanthine-guanine phosporibosyl transferase gene, CAD, adenosine deaminase gene, asparagine synthetase gene, numerous antibiotic resistance genes (tetracycline, ampicillin, kanamycin, neomycin, and the like), aminoglycoside phosphotransferase genes, hygromycin B phosphotransferase gene, and genes whose expression provides for the presence of a detectable product, either directly or indirectly, such as, for example, beta-galactosidase, GFP, and the like.

In addition to the at least one transcriptionally active gene, the portion of the transposon containing the inverted repeats also comprises at least one restriction endonuclease recognized site, e.g. restriction site, located between the flanking inverted repeats, which serves as a site for insertion of an exogenous nucleic acid. A variety of restriction sites are known in the art and include, but are not limited to: HindIII, PstI, SalI, AccI, HincII, XbaI, BamHI, SmaI, XmaI, KpnI, SacI, EcoRI, and the like. In some embodiments, the vector includes a polylinker, i.e. a closely arranged series or array of sites recognized by a plurality of different restriction enzymes, such as those listed above. In other embodiments, the inserted exogenous nucleic acid could comprise recombinase recognition sites, such as LoxP, FRT, or AttB/AttP sites, which are recognized by the Cre, Flp, and PhiC31 recombinases, respectively.

Where the source of hyperactive transposase is a nucleic acid that encodes the hyperactive transposase, the nucleic acid encoding the hyperactive transposase protein is generally part of an expression module, as described above, where the additional elements provide for expression of the transposase as required.

The present invention provides methods of integrating an exogenous nucleic acid into the genome of at least one cell of a multicellular or unicellular organism comprising administering directly to the multicellular or unicellular organism: a) a transposon comprising the exogenous nucleic acid, wherein the exogenous nucleic acid is flanked by one or more of any of the aforementioned inverted repeat sequences that are recognized by any of the aforementioned proteins; and b) any one of the aforementioned proteins to excise the exogenous nucleic acid from a plasmid, episome, or transgene and integrate the exogenous nucleic acid into the genome. In some embodiments, the protein of b) is administered as a nucleic acid encoding the protein. In some embodiments, the transposon and nucleic acid encoding the protein of b) are present on separate vectors. In some embodiments, the transposon and nucleic acid encoding the protein of b) are present on the same vector. When present on the same vector, the portion of the vector encoding the hyperactive transposase is located outside the portion carrying the inserted nucleic acid. In other words, the transposase encoding region is located external to the region flanked by the inverted repeats. Put another way, the transposase encoding region is positioned to the left of the left terminal inverted repeat or to the right of the right terminal inverted repeat. In the aforementioned methods, the hyperactive transposase protein recognizes the inverted repeats that flank an inserted nucleic acid, such as a nucleic acid that is to be inserted into a target cell genome.

In some embodiments, the vertebrate animal is a mammal, such as for example, a rodent (mouse or rat), livestock (pig, horse, cow, etc.), pets (dog or cat), and primates, such as, for example, a human.

The methods described herein can be used in a variety of applications in which it is desired to introduce and stably integrate an exogenous nucleic acid into the genome of a target cell. In certain embodiments, linear or circularized DNA, such as a plasmid, is employed as the vector for delivery of the transposon system to the target cell. In such embodiments, the plasmid may be administered in an aqueous delivery vehicle, such as a saline solution. Alternately, an agent that modulates the distribution of the vector in the multicellular or unicellular organism can be employed. For example, where the vectors comprising the subject system components are plasmid vectors, lipid-based such as a liposome, vehicles can be employed, where the lipid-based vehicle may be targeted to a specific cell type for cell or tissue specific delivery of the vector. Alternately, polylysine-based peptides can be employed as carriers, which may or may not be modified with targeting moieties, and the like (Brooks et al., J. Neurosci. Methods, 1998, 80, 137-47; and Muramatsu et al., Int. J. Mol. Med., 1998, 1, 55-62). The system components can also be incorporated onto viral vectors, such as adenovirus-derived vectors, sindbis-virus derived vectors, retrovirus-derived vectors, hybrid vectors, and the like. The above vectors and delivery vehicles are merely representative.

The elements of the transposase system are administered to the animal or in an in vivo manner such that they are introduced into germline of a parent animal. As the transposon is introduced into the cell “under conditions sufficient for excision and integration to occur,” the method can further include a step of ensuring that the requisite transposase activity is present in the target cell along with the introduced transposon. Depending on the structure of the transposon vector itself, such as whether or not the vector includes a region encoding a product having transposase activity, the method can further include introducing a second vector into the target cell that encodes the requisite transposase activity, where this step also includes an in vivo administration step.

The invention relates to a method of integrating a nucleic acid sequence into the germline of an animal comprising direct injection of a sterile saline solution comprising any composition disclosed herein.

The amount of vector nucleic acid that is introduced into the target cell varies depending on the efficiency of the particular animal protocol that is employed, such as transfer in a rat or mouse.

The particular dosage of each component of the system that is administered to the multicellular or unicellular organism varies depending on the nature of the transposon nucleic acid, e.g. the nature of the expression module and gene, the nature of the vector on which the component elements are present, the nature of the delivery vehicle and the like. For example, in mice where the transposase system components are present on separate plasmids which are intravenously administered to a mammal in a saline solution vehicle, the amount of transposon plasmid that is administered in many embodiments typically ranges from about 0.5 to 40 μg and is typically about 25 μg, while the amount of transposase encoding plasmid that is administered typically ranges from about 0.5 to 25 μg and is usually about 1 μg.

Once the vector DNA has entered the target cell in combination with the requisite transposase, the nucleic acid region of the vector that is flanked by inverted repeats, i.e. the vector nucleic acid positioned between the transposase-recognized inverted repeats, is excised from the vector via the provided transposase and inserted into the genome of the targeted cell. As such, introduction of the vector DNA into the target cell is followed by subsequent transposase mediated excision and insertion of the exogenous nucleic acid carried by the vector into the genome of the targeted cell.

The subject methods may be used to integrate nucleic acids of various sizes into the target cell genome. Generally, the size of DNA that is inserted into a target cell genome using the subject methods ranges from about 0.5 kb to 10.0 kb, usually from about 1.0 kb to about 8.0 kb.

The subject methods result in stable integration of the nucleic acid into the target cell genome. By stable integration is meant that the nucleic acid remains present in the target cell genome for more than a transient period of time, and is passed on a part of the chromosomal genetic material to the progeny of the target cell. The subject methods of stable integration of nucleic acids into the genome of a target cell find use in a variety of applications in which the stable integration of a nucleic acid into a target genome is desired. Applications in which the subject vectors and methods find use include, for example, research applications, polypeptide synthesis applications and therapeutic applications.

The present invention can be used in, for example, germline mutagenesis in a rat, mouse, or other vertebrate. In some embodiments, the composition comprises a nucleic acid sequence that encodes a hyperactive transposase.

The transposase system described herein can be used for germline mutagenesis in a vertebrate species. In some embodiments, the method of affecting germline mutations does not comprise any step including pronuclear injection.

Mutations (transposon insertions) can be detected by, for example, Southern blot and PCR. The specific insertion sites within each mutant animal can then be identified by, for example, linker-mediated PCR, inverse PCR, or other PCR cloning techniques. The Transposase transposon has a random distribution, in that it does not prefer any particular site in mammalian genomes when integrating. Thus, thousands of unique gene mutations are likely to be uncovered through Transposase-mediated germline mutagenesis. Some of the mutant animals identified via transposase-mediated mutagenesis can serve as valuable models for studying human disease.

The present invention also provides methods of generating a transgenic, non-human vertebrate comprising injection of a composition into the testis of a non-human vertebrate, such composition comprising a nucleic acid sequence encoding a transposase or any enzyme disclosed herein and a nucleotide sequence that, when integrated into the genome, modifies a trait in the transgenic, non-human vertebrate.

For production of transgenic animals containing two or more transgenes, such as in embodiments where the Transposase transposon and Transposase transposase components of the invention are introduced into an animal via separate nucleic acids, the transgenes can be introduced simultaneously using the same procedure as for a single transgene. Alternately, the transgenes can be initially introduced into separate animals and then combined into the same genome by breeding the animals. Alternately, a first transgenic animal is produced containing one of the transgenes. A second transgene is then introduced into fertilized ova or embryonic stem cells from that animal.

Transgenic mammals can be generated conventionally by introducing by microinjecting the above-described transgenes into mammals' fertilized eggs (those at the pronucleus phase), implanting the eggs in the oviducts of female mammals (recipient mammals) after a few additional incubation or directly in their uteri synchronized to the pseudopregnancy, and obtaining the offspring.

To find whether the generated offspring are transgenic, many procedures, such as dot-blotting, PCR, immunohistological, complement-inhibition analyses, and the like, can be used.

The transgenic mammals generated can be propagated by conventionally mating and obtaining the offspring, or transferring nuclei (nucleus transfer) of the transgenic mammal's somatic cells, which have been initialized or not, into fertilized eggs of which nuclei have previously been enucleated, implanting the eggs in the oviducts or uteri of the recipient mammals, and obtaining the clone offspring.

Transformed cells and/or transgenic organisms, such as those containing the DNA inserted into the host cell's DNA, can be selected from untransformed cells and/or transformed organisms if a selectable marker is included as part of the introduced DNA sequences. Selectable markers include, for example, genes that provide antibiotic resistance; genes that modify the physiology of the host, such as for example green fluorescent protein, to produce an altered visible phenotype. Cells and/or organisms containing these genes are capable of surviving in the presence of antibiotic, insecticides or herbicide concentrations that kill untransformed cells/organisms or producing an altered visible phenotype. Using standard techniques known to those familiar with the field, techniques such as, for example, Southern blotting and polymerase chain reaction, DNA can be isolated from transgenic cells and/or organisms to confirm that the introduced DNA has been inserted.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted. All journal articles, patent applications, issued patents or other citations are incorporated by reference herein in their entireties.

### Examples

### Results

### Seminiferous Tubule Injections.

A solution of DNA plus Lipofectamine, the same solution routinely in our laboratory to transfect rat spermatogonial stem cells in vitro, was injected into the seminiferous tubules of 18 weanling male rats via the rete testis. This DNA consisted of 6 μg of the plasmid PB-TSV, which contains a transposon carrying the genes for neomycin resistance, puromycin resistance and copGFP, either alone or with an additional 1 μg of the plasmid sPBo, which contains a hyperactive piggyBac transposase gene. This was done using various ratios of the Plus Reagent and LTX reagents supplied by the manufacturer to determine whether altering the ratios might alter transfection efficiency. Animals were allowed to age 6-8 weeks to reach sexual maturity, at which point 10 were sacrificed and epididymal sperm was isolated from the animals and assayed for the presence of the copGFP transgene by PCR. The remaining animals were allowed to mate for one week, and at this point 5 were sacrificed to assay these sperm for the transgene. The remaining 3 animals were allowed to mate for another two weeks before they too were sacrificed and assayed for the transgene in the sperm. The results are summarized in Table 1. Of the 18 animals, three had transgene detectable in the epididymal sperm, and an intersecting subset of three animals produced pups.

We then assayed DNA from the pups produced by the three animals, 524013, 523977 and 523978. Interestingly, when these animals were sacrificed and their sperm assayed for the presence of transgene DNA by PCR, none could be detected in the sperm of animal 524013, while only weak signal was detected in the sperm of the other two. Animal 523978, which was injected with the transposon alone, only produced a single pup, which died and was not recovered and was therefore not genotyped. The other two animals, which were injected by a combination of transposon plus transposase DNAs, produced a viable litter apiece. DNA was isolated from ear punch biopsies of these animals, and this was again assayed for the copGFP transgene by PCR. Full results are summarized in Table 2. The efficiency of transfer of DNA to the F1 animals was impressive: 8 of 9 animals in the first litter carried the copGFP transgene, while 7 of 12 in the second litter were similarly positive. The animals were also assayed for the presence of the sPBo transposase; none were positive for the helper plasmid.

These results demonstrate that DNA injected into the seminiferous tubules can indeed be transmitted to the embryos, presumably via the sperm. Interestingly, a high percentage of the offspring of animal 524013 were transgenic although no transgene could be found in the sperm of this animal when it was collected subsequent to the siring of this litter. This suggests that the injection of the DNA solution into the seminiferous tubes results in a transient pool of DNA that can be transferred to the sperm, but that DNA pool is eventually depleted. In this context, it is interesting to speculate that failure to find DNA in the epididymal sperm of the majority of these animals might reflect the earlier depletion of this DNA pool. Additionally, the observation that F1 pups contained transposon DNA but did not contain any of the helper plasmid sPBo encoding the transposase suggests that the transgene was inserted into the genome of F1 animals via the action of the transposase, rather than by random integration, although it must be noted that the helper plasmid was provided at ⅙ the concentration of the transposon plasmid.

DNA copy numbers were determined for each of the putative transgenic F1 founders by qPCR. Copy numbers ranged from 0.02 copies per genome to 4.25 copies per genome (data not shown). These data demonstrated that many of these animals were likely to be mosaic, but that some were likely to be germline transgenics.

Several of the putatively transgenic F1 animals with higher copy numbers were then mated with wildtype animals and in order to pass the transgene on to the F2 generation, in order to demonstrate stable germline trans genesis.

Three transgenic F1 offspring of animal 523977 and one F1 of animal 524013 with transposon copy numbers ranging from 0.3 copies per genome to 4.3 copies per genome were mated with wildtype animals. Only two of the 523977 F1s have produced offspring to date. These animals, 537453 (0.3 copies per genome) and 531469 (4.3 copies per genome) produced 13 pups each in their first litters. Again, DNA was isolated from earpunch biopsies from these animals, and the DNA was assayed for presence of the copGFP transgene by PCR. The 3 of thirteen offspring from 537453 carried the transgene, while animal 531469 produced 9 of thirteen pups carrying the transgene.

These latter results conclusively demonstrate stable germline transmission via this method. The results are also consistent with the copy number analysis, since the animal with <1 copy of the transposon in the genome gave fewer than 50% transgenic F2s, suggesting some degree of germline mosaicism, while the animal carrying 4.3 copies of the transposon produced well over 50% transgenic F2 animals, consistent with either multiple insertions in the germline or a germline population consisting of a mix of cells with different insertions.

These data, however, do not conclusively demonstrate that the transgenes have been integrated through the action of the transposase as opposed to the random integration of the transposon plasmid. Since no viable animals were obtained from the control animal injected with the transposon in the absence of transposase, we determine the baseline integration rates of the transposon plasmid. Again, it is suggestive that the helper plasmid was not integrated, which can only occur via random insertion, and very few publications have reported transmission of TMGT-delivered transgenes to F2 animals. We will be able to definitively prove transpositional insertion by characterizing the insertion site of the DNA.

If the transgene has been inserted via transposition, sequencing outward from just within the transposon boundaries should show transposon ITR sequences followed by a TTAA and then rat genomic DNA sequences, whereas if the transgene has been inserted by the random integration of the transposon plasmid, plasmid sequences should exist outside of the ITRs. We will sequence outward from the transposon boundaries to show ITR sequences followed by a TTAA and then rat genomic DNA sequences.

### Testicular Body Injections.

A solution of plasmids plus SuperFect was injected into the body of the testis of adult male animals using a small syringe and a 30 gauge needle. In each testis, 9 μg of transposon plasmid with injected, either alone or mixed with 1 μg sPBo (transposase) plasmid. One of two transposons were utilized, either PB-TSV or PB-IFNg. The former was the same transposon used in the injections of DNA into the tubules, while the latter carries an IFNg transgene and a copGFP reporter transgene. A total of six animals were injected, two with PB-TSV alone, two with PB-TSV plus sBPo and two with PB-IFNg. Five days after DNA injection, the animals were allowed to mate for two weeks, and then they were sacrificed and assayed for the presence of DNA in epididymal sperm by PCR detection of the copGFP transgene.

Treatments and results are summarized in Table 3. Transgene DNA was detected in sperm from three of the animals: 532801, 532803 and 532805. Mating of the six animals resulted in 4 productive breedings from animals 532800, 532802, 532804 and 532805.

The F1 animals were then assayed for presence of the transgene, and the vast majority of the pups were transgene positive. Results were collected that show the PCR analysis of DNAs from the pups from animal 532803 injected with transposon PB-TSV alone and from animal 532800, injected with PB-TSV plus sPBo; in these two cases the transgene can be detected in every animal. The results also show the analysis of the pups generated from animal 532802, injected with PB-IFNg plus sPBo where 4 of 12 animals were transgenic, and from animal 532805, also injected with PB-TSV plus sPBo, where 7 of 12 pups are transgenic. Note that once again, an animal with no detectable transgene DNA in epididymal sperm (animal 532800) at the end of mating nonetheless was able pass on the transgene, again suggesting that the injected DNA exists as a transient pool that is eventually depleted.

These results confirmed the results of Yonezawa, (2001) who reported that this method was very efficient in transferring DNA to F1 animals. It also demonstrated that the DNA must be transferred via mature sperm, since the time for mating and DNA transfer was less than the time for a spermatogenic cycle to occur in the rat. Indeed, animals were born approximately a month after the injection date, and given the 21-day gestation period of the rat and 5-day delay between DNA injection and mating, the animals must have mated almost immediately.

Although Yonezawa (2001) was unable to demonstrate transmission of transgene DNA to the F2 generation, we then crossed some of the F1 transgenic animals (with apparently higher copy number, based on PCR band intensity) to wildtype animals, obtained F2s that we again assayed by PCR. The results were dramatic. Offspring of an F1 animal generated with PB-TSV alone had no detectable copGFP signal (with the exception of a very faint band in one lane visible only when the gel was overexposed); meanwhile all F2 animals from transposon plus transposase animals were transgenic. These results very strongly indicate that the transposase gene is being expressed and is catalyzing the insertion of the transposon early enough in embryo so that transposition of the transposons takes place into the genome of the germline lineage.

To further demonstrate that the transposon DNA was inserting via transposition, DNA of an F1 transgenic animal and one of its transgenic F2 offspring were subjected to splinkerette analysis, which characterizes the insertion site of the transposon. In this assay, genomic DNA is digested completely with a 4-base cutting restriction endonuclease. DNA “splinkers”, small DNA tails, are then ligated onto the ends of the resulting fragments. At the completion of the ligation, the DNA fragments will include fragments of DNA including the ends of the transposon and a small amount of flanking region with splinkers ligated to both ends. The ligated DNA mix is again digested to remove the internal splinker from the transposon end fragments, and the mix is then PCR amplified using primers that anneal to the transposon sequence and to the splinker, thus amplifying the end of the transposon and the flanking genomic DNA. The PCR products are then cloned and sequenced, and the resulting sequence compared to the genomic sequence to identify the site(s) of transposon insertion. Results were collected that show the gel of the splinkerette products of F1 PB-IFNg+sPBo transgenic animal and transgenic F2 offspring. The F1 animal appears to have four separate insertion sites, as indicated by the presence of four bands, while one of these bands was transmitted to the F2 animal. One of the F1 bands conclusively maps to rat Chromosome 8 and is definitively a transposon insertion event by sequence. The F2 fragment is also a transposon insertion, but its map position cannot be definitively identified. The other two splinkerette bands have yet to be subcloned and sequenced.

### DISCUSSION

Taken together, our results demonstrate our ability to create stable germline transgenic rats via the injection of transposon plus transposae-encoding DNA combined with transfection reagents into the testis of rats. Of the two approaches reported here, the injection of DNA complexed with SuperFect directly into the body of the testis is much preferred over the injection of the DNA/Lipofectamine solution into the seminiferous tissues. First, it is faster, since it requires no wait for the injected animals to mature. In addition, the procedure itself is also much simpler, since only a small incision is made to access the testis, which is then injected, as opposed the exteriorization of the testis and the precise and somewhat difficult injection of the DNA into the rete testis.

The utilization of sperm to deliver DNA to the embryo has been a goal since the 1989, when Laivatrano et al reported simple and efficient transgenesis accomplished via the incubation of mouse sperm with a DNA solution followed by IVF, resulting in an impressive 30% transgenic animals. This work was widely criticized as irreproducible, and Brinster et al. (1989) published a refutation of this work that not only discussed negative data from a number of independent research group but also reported that the technique resulted in zero transgenic animals out of 890 pups. Nonetheless, numerous further attempts have been made, with some success being obtained in a variety of mammalian species by introducing the DNA to the sperm in combination with transfection reagents or electroporation to produce animals that indeed carry the transgene. However, these transgenic F1 animals never (with the exception of the initial Laivatrano report and a single follow-on paper (Laivatrano et al, 1998) transmitted the transgene to their offspring. Apparently, the transgenes either exist as episomes that are eventually lost, or are not incorporated into the germline.

There is, however, one exception to the failure of SMGT to produce stable germline transgenic animals: when DNA is incubated with sperm that have been disrupted with detergent or freeze-thaw cycles, stable germline transgenesis can be accomplished by Intracytoplasmic Sperm Injection (ICSI) in which the sperm or sperm heads are physically microinjected into oocytes. However, the technique once again introduces the necessity for using large numbers of animals for oocyte production and a complex and technically difficult process, obviating the simplicity promised by SMGT. Nonetheless, it has been successfully used TMTG is a variation of SMGT where DNA is applied to the testis, where it then makes its way into the sperm. Various methodologies have been reported, including injecting the DNA, usually complexed with liposomes, into the body of the testis or into the seminiferous tubules; sometimes an electric pulse is then applied to the testis in an attempt to increase DNA incorporation rates via in vivo electroporation. The goal is usually to stably integrate the transgene into the spermatogonial cells that will then produce transgenic sperm, but all results indicate that the DNA somehow makes its way to the sperm and is carried into the oocyte during fertilization as it is with SMGT. In any event, there are many reports of success in a variety of species, but again, with the exception of only two reports (Sato et al. 1999; Miao and Zhang, 2011), as with SMGT the transgene is transmitted to the F1 offspring but it is never transmitted from there to the F2. At best, the reports of stable transgene integration into the germline can only reflect the extraordinarily rare nature of this event using this method.

The transfer of DNA to F1 animals, however, can be relatively efficient, as exemplified by the report of Yonezawa et al (2001). In this work, the authors injected DNA into the body of the testis as a DNA-liposome complex. They surveyed an number of commercial transfection reagents, and demonstrated that using SuperFect, the injected DNA could be detected in epididymal sperm from all animals (all this was a small number, n=2 or 3) from 1 days 2 weeks after injection. Furthermore, it could be detected in 82% of all morulae (and expression of the transgene, a GFP reporter gene, could be observed), though this number dropped to 22% of 14 d.p.c. fetuses, 5% of neonates and pre-weanlings, and 4% of one-month old pups. Transgenesis of post-implantation stages was only demonstrated by PCR, and transgenic fetuses and animals were demonstrably mosaic. Although the authors held forth the promise that this method would lead to a simple method of transgenic production, this has not been the case. Indeed, the loss of the transgene through development suggest a general failure of transgenes delivered by this method to incorporate into the genome of the germline lineage.

The piggyBac transposon system is a powerful tool for the introduction of DNA into the genome of cells, and it has been applied to make transgenic mice at high efficiency (Katter et al., 2013; Rostovskaya et al, 2013) by the coinjection of a transposon plus transposase into one-cell embryos utilizing the same technique by which transgenic DNA constructs are usually introduced. Interestingly, in one other case of transgene production (Marh et al, 2012), ICSI-transgenesis was used to deliver a piggyBac transposon/transposase vector to oocytes, resulting in much improved efficiency of transgenic animal production. In all these cases, the transposase acts to increase the rate of the insertion of the transposon-based transgene into the genome of the egg over the insertion rate of naked DNA alone.

The methodology we have demonstrated here now combines the simplicity and efficiency of DNA delivery to the oocyte via TMTG, but it also harnesses the ability of the piggyBac transposase to efficiently insert a transposon carrying a transgene into the genome. Furthermore, although we have demonstrated this in rats, TMTG has also been use to produce F1 transgenics in a variety of other mammalian species. In addition, since sperm alone are capable of also transmitting DNA to the oocyte after artificial insemination, again in a number mammalian and even non-mammalian species, one should be able to apply this method via SMGT.

It has not escaped our attention that other types of genes whose products can modify the genome can potentially be delivered to the oocyte via this method. Thus, one would expect that not only could other transposase/transposon systems such as Transposase be used, but genes encoding the targeted double-stranded nucleases such as Zinc Finger Nucleases, TALENs, Meganucleases, or CRISPR, or recombinases such as Cre or FLP, integrases such as phiC3, and a host of other DNA or chromatin-modification enzymes such as DNA methylases, histone deacetylases and the like.

Thus, there is great promise that we can harness this technique to provide vastly simplified genome modification in a large number of species, using it not only to easily introduce transgenes but to mobilize or excise transposons, perform targeted mutagenesis, manipulate floxed DNA, perform recombinase-mediated cassette exchange, or any of the other myriad methods by which we now modify the genome using methods that are much more complex. We call this generalized technology Sperm-mediated Genome Engineering, or SGM.

TABLE 1
Animals into which a DNA: Lipofectamine solution was injected into the rete testis.

| | | | | | | | | | |
|---|---|---|---|---|---|---|---|---|---|
| | | | | | | | DNA in | | |
| | | | Ratio | DNA | | | Epididymal | Pups | Transgenic |
| Date Inj | Animal | DOB | PlusReag:LTX | Condition | Right | Left | Sperm? | Produced? | Pups? |

Animals sacrificed at 6-8 weeks post-implantation and assayed for transgene DNA in sperm

| | | | | | | | | | |
|---|---|---|---|---|---|---|---|---|---|
| May 9, 2012 | 509112 | Apr. 16, 2012 | 2:4 | Transposon + | Miss | 95% | No | NA | NA |
| | | | | Transposase | | | | | |
| May 9, 2012 | 509113 | Apr. 16, 2012 | 2:4 | Transposon + | 80% | Miss | No | NA | NA |
| | | | | Transposase | | | | | |
| May 9, 2012 | 509114 | Apr. 16, 2012 | 2:4 | Transposon + | 80% | 85% | No | NA | NA |
| | | | | Transposase | | | | | |
| May 10, 2012 | 519290 | Apr. 16, 2012 | 1.5:3 | Transposon + | 10% | Miss | No | NA | NA |
| | | | | Transposase | | | | | |
| May 18, 2012 | 519291 | Apr. 25, 2012 | 2:4 | Transposon | 25% | 100% | No | NA | NA |
| May 18, 2012 | 524006 | Apr. 25, 2012 | 2:4 | Transposon | 80% | 100% | No | NA | NA |
| May 18, 2012 | 524007 | Apr. 25, 2012 | 2:4 | Transposon | 100% | 95% | No | NA | NA |
| May 18, 2012 | 519294 | Apr. 25, 2012 | 1.5:3 | Transposon + | 80% | Miss | No | NA | NA |
| | | | | Transposase | | | | | |
| May 25, 2012 | 524009 | May 2, 2012 | 3:6 | Transposon + | 70% | 30% | YES | NA | NA |
| | | | | Transposase | | | | | |
| May 25, 2012 | 524010 | May 2, 2012 | 3:6 | Transposon | 90% | 100% | No | NA | NA |

Animals mated one week then sacrificed for sperm analysis

| | | | | | | | | | |
|---|---|---|---|---|---|---|---|---|---|
| May 25, 2012 | 524008 | May 3, 2012 | 1.5:3 | Transposon + | 80% | Miss | No | No | NA |
| | | | | Transposase | | | | | |
| Jun. 1, 2012 | 524013 | May 10, 2012 | 1.5:3 | Transposon + | 70% | 75% | NO | YES | YES |
| | | | | Transposase | | | | | |
| Jun. 1, 2012 | 524014 | May 10, 2012 | 3:6 | Transposon + | 100% | 100% | No | No | NA |
| | | | | Transposase | | | | | |
| Jun. 1, 2012 | 524015 | May 10, 2012 | 3:6 | Transposon | 85% | 60% | No | No | NA |
| Jun. 22, 2012 | 523994 | May 29, 2012 | 1.5:3 | Transposon + | 90% | Miss | No | No | NA |
| | | | | Transposase | | | | | |

Animals mated for 3 weeks and then sacrificed for sperm analysis

| | | | | | | | | | |
|---|---|---|---|---|---|---|---|---|---|
| Jun. 15, 2012 | 523977 | May 22, 2012 | 3:6 | Transposon + | 100% | 100% | YES | YES | YES |
| | | | | Transposase | | | | | |
| Jun. 15, 2012 | 523978 | May 22, 2012 | 3:6 | Transposon | 40% | 100% | YES | YES | ? |
| Jun. 22, 2012 | 523993 | May 29, 2012 | 1.5:3 | Transposon + | 10% | 60% | No | No | NA |
| | | | | Transposase | | | | | |

TABLE 2
Animals in which DNA/SuperFect was injected into the
body of the testis.

| Date Inj | Animal | DOB | DNA Injected | Pups Born |
|---|---|---|---|---|
| Sep. 10, 2012 | 532801 | Jul. 13, 2012 | PB-TSV 9 μg | |
| Sep. 10, 2012 | 532800 | Jul. 13, 2012 | PB-TSV 9 μg + | Oct. 9, 2012 |
| | | | 1 μg | |
| Sep. 10, 2012 | 532802 | Jul. 13, 2012 | PB-IFNg | Oct. 9, 2012 |
| | | | 9 μg + | |
| | | | sPBo 1 μg | |
| Sep. 10, 2012 | 532803 | Jul. 13, 2012 | PB-TSV 9 μg | Oct. 11, 2012 |
| Sep. 10, 2012 | 532805 | Jul. 13, 2012 | PB-TSV 9 μg + | Oct. 11, 2012 |
| | | | sPBo 1 μg | |
| Sep. 10, 2012 | 532804 | Jul. 13, 2012 | PB-IFNg | |
| | | | 9 μg + | |
| | | | sPBo 1 μg | |

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method of integrating an exogenous nucleic acid into the genome of at least one cell of an animal comprising administering a composition directly to the testis of the animal, wherein the composition comprises
a) a transposon comprising an exogenous nucleic acid, wherein the exogenous nucleic acid is flanked by a first inverted repeat sequence comprising a sequence at least about 90% sequence identity to one ITR of a transposase disclosed herein and/or a second inverted repeat sequence comprising a sequence at least about 90% sequence identity to a known ITR of a transposase disclosed herein; and
b) a nucleic acid encoding any transposase disclosed herein, according to any one to excise the exogenous nucleic acid from a plasmid, episome, or transgene and integrate the exogenous nucleic acid into the genome of the animal.

2. The method according to claim 1 wherein the transposon and nucleic acid encoding the transposase of b) are present on separate vectors.

3. The method according to claim 1 wherein the transposon and nucleic acid encoding the transposase of b) are present on the same vector.

4. The method according to claim 1, wherein the animal is a vertebrate.

5. The method according to claim 4 wherein the vertebrate animal is a mammal.

6. The method according to claim 1, wherein the step of administering is administering via injection with a composition comprising the nucleic acid sequence (a) and (b), and wherein the composition is sterile and pyrogen-free.

7. The method according to claim 1, wherein the exogenous nucleic acid comprises a gene expressible in a mammal.

8. A method of generating a non-human, transgenic animal comprising a germline mutation comprising administering directly to the testis of the animal:
a composition comprising:
a) a nucleic acid comprising an exogenous nucleic acid; and
b) a nucleic acid encoding a genetic modification enzyme selected from: a nuclease, a recombinase, a methylase, a deacetylase, and an integrase; and wherein the nucleic acid encoding the modification enzyme is free of a nucleic acid sequence encoding transposase.

9. A method of increasing the efficiency of germline mutagenesis in an animal comprising administering the composition of claim 1 directly into the testis of an animal.

10. The method of claim 1, wherein the injection is administered into the seminiferous tubules or the retes testis.

11. A method of increasing the efficiency of germline transmission of a mutation in an animal from an F2 generation, the method comprising administering a composition via direct injection into the testis of a parent animal to create an animal from an F1 with a mutated germline; breeding the animal from an F1 with a mutated germline with another animal from the same species to create an animal from the F2 generation with the mutation; wherein the composition comprises:
a) a nucleic acid comprising an exogenous nucleic acid; and
b) a nucleic acid encoding a genetic modification enzyme selected from: a nuclease, a recombinase, a methylase, a deacetylase, transposase and an integrase.

12. The method of claim 11, wherein the animals from each generation are transgenic animals.

13. A method of increasing the efficiency of germline transmission of a mutation in an animal the method comprising administering a composition via direct injection into the testis of a parent animal, wherein the composition comprises:
a) a transposon comprising an exogenous nucleic acid, wherein the exogenous nucleic acid is flanked by a first inverted repeat sequence comprising a sequence at least about 90% sequence identity to one ITR of a transposase disclosed herein and/or a second inverted repeat sequence comprising a sequence at least about 90% sequence identity to a known ITR of a transposase disclosed herein; and
b) a nucleic acid encoding any transposase disclosed herein, according to any one to excise the exogenous nucleic acid from a plasmid, episome, or transgene and integrate the exogenous nucleic acid into the genome of the animal.

14. The method of claim 11, wherein the efficiency of mutation frequency is increased by at least 10%, 20%, 30%, 40%, or 50% as compared to direct injection of a composition free of any genetic modification enzyme.

15. A method of generating a non-human, transgenic animal comprising: introducing a nucleic acid molecule comprising an exogenous nucleic acid and a genetic modification enzyme or a nucleic acid encoding a genetic modification enzyme into the testis of an animal via direct injection; wherein the genetic modification enzyme is selected from: a nuclease, a recombinase, a methylase, a deacetylase, a transposase and an integrase.

16. A method of generating a non-human, transgenic animal comprising: mixing a composition with an isolated sperm cell ex vivo; and either artificially inseminating a recipient animal or performing in vitro fertilization with the isolated sperm cell, wherein the composition comprises:
a) a nucleic acid comprising an exogenous nucleic acid; and
b) a nucleic acid encoding a genetic modification enzyme selected from: a nuclease, a recombinase, a methylase, a deacetylase, transposase and an integrase.

17. A method of increasing the efficiency of germline transmission of a mutation in an F1 or F2 generation of an animal, the method comprising: mixing a composition with an isolated sperm cell ex vivo; and either artificially inseminating a recipient animal or performing in vitro fertilization with the isolated sperm cell, wherein the composition comprises:
a) a nucleic acid comprising an exogenous nucleic acid; and
b) a genetic modification enzyme or a nucleic acid encoding a genetic modification enzyme, wherein the enzyme selected from: a nuclease, a recombinase, a methylase, a deacetylase, a transposase and an integrase.

18. The method of claim 17 wherein the efficiency of mutation frequency is increased by at least 10%, 20%, 30%, 40%, or 50% as compared to artificially inseminating a recipient animal or performing in vitro fertilization after mixing sperm with a composition that does not comprise a nucleic acid encoding a genetic modification enzyme or a genetic modification enzyme.

19. The method of claim 8, wherein the genetic modification enzyme is a nuclease.

20. The method of claim 19, wherein the nuclease is a zinc finger nuclease, transcription activator-like effector nucleases (TALEN), a meganuclease, or a clustered regularly interspaced short palindromic repeats (CRISPR) nuclease.
